(19) United States
(12) Patent Application Publication
(10) Pub. No.: US 2020/0279633 A1
(43) Pub. Date: Sep. 3, 2020

(54) SYSTEM AND METHOD FOR ASSISTING TARGET PERSON IN BEHAVIOR CHANGE AND HABITUATION

(71) Applicant: Hitachi, Ltd., Tokyo (JP)

(72) Inventors: Mami SASAKI, Tokyo (JP); Eitaro ITO, Tokyo (JP); Tatsuya TOKUNAGA, Tokyo (JP); Daisuke NOGAMI, Tokyo (JP); Satomi HORI, Tokyo (JP); Ryota NIIZEKI, Tokyo (JP); Yuta TSURUGA, Tokyo (JP); Kozue KANO, Tokyo (JP); Eriko UEGAKI, Tokyo (JP); Eizaburou TAKEGAMI, Tokyo (JP)

(21) Appl. No.: 16/793,067

(22) Filed: Feb. 18, 2020

(30) Foreign Application Priority Data
Mar. 1, 2019 (JP) 2019-037199

(51) Int. Cl. G16H 20/30; G16H 50/70; G16H 10/20

(52) U.S. Cl. CPC G16H 20/30; G16H 10/20; G16H 50/70

(57) ABSTRACT

A system analyzes behavior data obtained by measuring behaviors of a plurality of target persons, and based on a result of the analysis, defines a stage indicator, which is an indicator as a criterion of a plurality of stages via which a behavior as a target of habituation is aimed at step by step, and each of the plurality of stages following the stage indicator. For each pair of adjacent stages, the system identifies a gap between two stages constituting the pair. For each stage pair, the system identifies a reason/measure that is at least one of a reason for existence of the identified gap and a measure for causing a target person belonging to a lower stage to make a behavior change to shift to a higher stage, from relationship information in which a relationship between the gap and the reason/measure is defined.

### CROSS-REFERENCE TO PRIOR APPLICATION

This application relates to and claims the benefit of priority from Japanese Patent Application number 2019-037199, filed on Mar. 1, 2019 the entire disclosure of which is incorporated herein by reference.

### BACKGROUND

The present invention relates to a novel technique for assisting a target person in behavior change and habituation by using a computer.

In recent years, efforts to use approaches based on a theory of behavioral science, which scientifically study human behavior, to develop services have expanded to various fields, including public policies, retail business, medical care, and environment.

In such efforts, a target is set, and it is considered what approach is taken to achieve the target. For example, a decision-making support technique disclosed in Patent Literature 1 is among techniques for assisting in target achievement.

Patent Literature 1: Japanese Patent Laid-Open No. 2018-109890

### SUMMARY

For an approach toward target achievement, for example, a generally used method is issuance of a persuasive message appealing to social norms, or grant of an incentive as a system of benefiting from a certain behavior.

Such a method has a problem that even though effects are seen in an initial period following introduction, the effects diminish after a lapse of some time period, so that continued use of the method fails.

Accordingly, in some fields such as health fields, a method known as the Stages of Behavior Change Model is adopted in some cases. The “Stages of Behavior Change Model” is a method in which stages of behavior change of a target person are divided into five stages of behavior change including “pre-contemplation”, “contemplation”, preparation”, “action”, and “maintenance”, and the target person, by being given step-by-step stimulations tailored to each stage of behavior change, is encouraged to make a behavior change to shift to a higher stage of behavior change and to become habituated.

However, it greatly depends on knowledge and know-how of a creator of a model of the stages of behavior change whether or not the model is appropriate. In fields where the “Stages of Behavior Change Model” method is not considered, knowledge or know-how to create such a model is not necessarily available. Accordingly, in such fields, measures for encouraging behavior change tend to be designed to directly aim at a final goal although step-by-step stimulations are necessary.

A system according to an embodiment of the present invention analyzes behavior data including various data obtained by measuring behaviors of a plurality of target persons, and based on a result of the analysis, defines a stage indicator, which is an indicator as a criterion of a plurality of stages via which a behavior as a target of habituation is aimed at step by step, and each of the plurality of stages following the stage indicator. For each pair of adjacent stages among the plurality of stages, the system identifies a stage gap, which is a gap between two stages constituting a stage pair. For each stage pair, the system identifies a gap reason/shift measure that is at least one of a gap reason, which is a reason for existence of the identified stage gap, and a shift measure, which is a measure for causing a target person belonging to a lower stage to make a behavior change to shift to a higher stage, from relationship information in which a relationship between the stage gap and the gap reason/shift measure is defined. The system executes result processing, which is processing related to the gap reason/shift measure identified for each stage pair.

In various fields, it can be expected that assisting a target person in behavior change and habituation is technically achieved.

### BRIEF DESCRIPTION OF THE DRAWINGS

FIG. 1 shows an outline of a first embodiment;

FIG. 2 shows an example of an entire configuration of a system according to the first embodiment;

FIG. 3 shows an example of a structure of a field pattern table;

FIG. 4 shows an example of a structure of a factor table;

FIG. 5 shows an example of a structure of a cognitive bias table;

FIG. 6 shows an example of a structure of a measure table;

FIG. 7 shows an example of a flow of entire processing performed in the first embodiment;

FIG. 8 shows an example of a behavior design canvas sheet screen;

FIG. 9 shows an example of a customer journey map screen;

FIG. 10 shows an example of an entire configuration of a system according to a second embodiment;

FIG. 11 shows an example of a flow of entire processing performed in the second embodiment; and

FIG. 12 shows an example of an individual report.

### DESCRIPTION OF EMBODIMENTS

In the description below, “interface apparatus” may include one or more interface devices. The one or more interface devices may be at least one of the following.
One or more I/O (Input/Output) interface devices. Each I/O (Input/Output) interface device is an interface device supporting at least one of an I/O device and a remote computer for display. An I/O interface device supporting the computer for display may be a communication interface device. At least one I/O device may be any one of user interface devices, for example, an input device such as a keyboard or a pointing device and an output device such as a display device.
One or more communication interface devices. The one or more communication interface devices may be one or more communication interface devices of a same type (for example, one or more NICs (Network Interface Cards)), or may be two or more communication interface devices of different types (for example, an NIC and an HBA (Host Bus Adapter)).

In the description below, “memory” may be a memory different from an NVM within an NVM drive, may include one or more memory devices, and may typically be one or more primary storage devices. At least one memory device in the memory may be a volatile memory device, or may be a non-volatile memory device.

In the description below, “persistent storage apparatus” includes one or more persistent storage devices. Each persistent storage device is typically a non-volatile storage device (for example, a secondary storage device), more specifically, for example, an HDD (Hard Disk Drive) or an SSD (Solid State Drive).

In the description below, “storage apparatus” may be at least the memory of the memory and the persistent storage apparatus.

In the description below, “processor” includes one or more processor devices. At least one processor device is typically a microprocessor device such as a CPU (Central Processing Unit), but may be another type of processor device such as a GPU (Graphics Processing Unit). At least one processor device may be a single core or may be a multi-core. At least one processor device may be a processor core. At least one processor device may be a processor device in a broad sense, such as a hardware circuit (for example, an FPGA (Field-Programmable Gate Array) or an ASIC (Application Specific Integrated Circuit)) that executes part or a whole of processing.

In the description below, an expression “xxx table” is used to describe information indicating that an output is derived from an input, and such information may be data in any structure, or may be a learning model such as a neural network that produces an output corresponding to an input. Accordingly, “xxx table” can be expressed as “xxx information”. In the description below, a structure of each table is an example, and a single table may be divided into two or more tables, and all or some of two or more tables may be a single table.

In the description below, an expression “kkk section” is used to describe a function, and a function may be implemented by a processor executing one or more computer programs, or may be implemented by one or more hardware circuits (for example, FPGA or ASIC). When a function is implemented by a processor executing one or more programs, the function may be at least part of the processor because predetermined processing is performed while a storage apparatus and/or an interface apparatus or the like is used as appropriate. Processing that is described by using a function as a subject of a sentence may be processing performed by the processor or an apparatus including the processor. The program may be installed from a program source. The program source may be, for example, a program distribution computer or a computer-readable storage medium (for example, a non-transient storage medium). A description of each function is an example, and a plurality of functions may be combined into a single function, and a single function may be divided into a plurality of functions.

In the description below, a system assisting a target person in behavior change and habituation is referred to as “assistance system”. The “assistance system” may be one or more physical computers, may be a software-defined system implemented by at least one physical computer executing predetermined software, or may be a system implemented on a cloud stack (typically, a plurality of types of computing resources including a processor and a storage apparatus). For example, when a computer includes a display device and the computer displays information on the own display device, the computer may be the assistance system. For example, when a first computer (for example, a server) transmits output information to a remote second computer (a computer for display (for example, an administrator terminal, which will be described later)) and the computer for display displays the information (when the first computer displays information on the second computer), at least the first computer of the first computer and the second computer may be the assistance system. In other words, “displaying output information” by the assistance system may be “displaying output information on a display device included in a computer”, or may be “transmitting output information to a computer for display” by a computer (in the latter case, the output information is displayed by the computer for display).

In the description below, when elements of a same type are described indistinguishably, a common part of reference signs is used, and when elements of a same type are described distinguishably, reference signs are used, in some cases. For example, when stages are described indistinguishably, “stage 50” is used, and when stages are described distinguishably, the stages are referred to as “stage 50A”, “stage 50B”, and the like, in some cases.

In the description below, “data set” is a single set of logical electronic data viewed from a program such as an application program, and may be any of, for example, records, a file, a key-value pair, a tuple, and the like.

Hereinafter, several embodiments will be described.

### First Embodiment

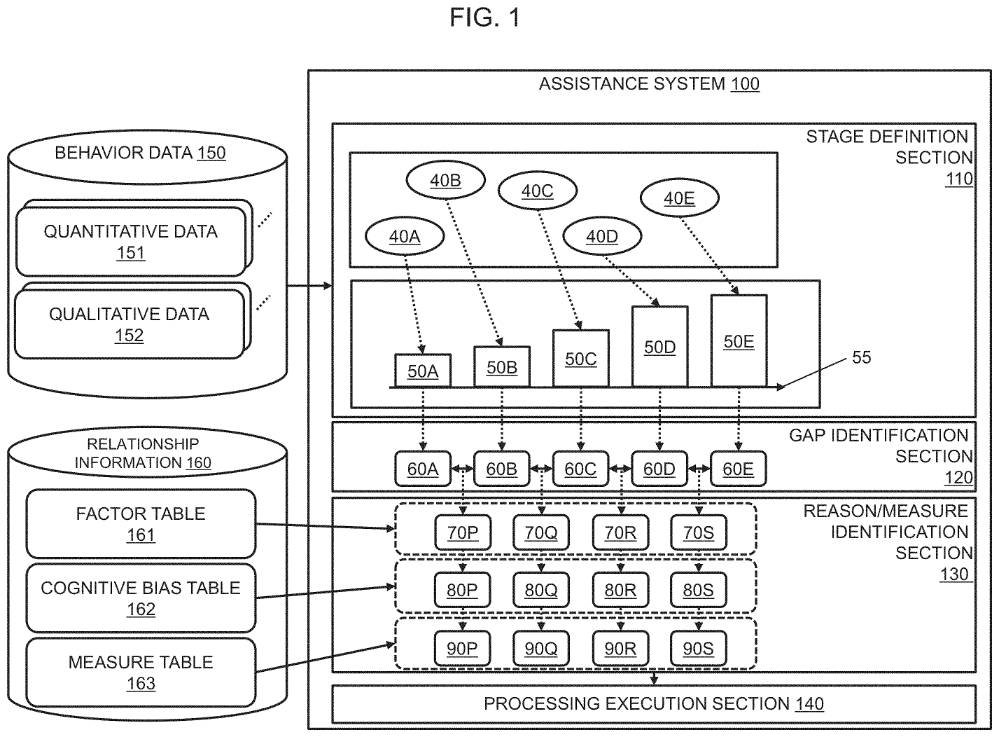

FIG. 1 shows an outline of a first embodiment.

An assistance system 100 includes a stage definition section 110, a gap identification section 120, a reason/measure identification section 130, and a processing execution section 140.

The stage definition section 110 defines a stage indicator 55, which is an indicator as a criterion of a plurality of stages 50 via which a behavior as a target of habituation is aimed at step by step, and each of the plurality of stages 50 following the stage indicator 55, based on behavior data 150 including various data obtained by measuring behaviors of a plurality of target persons. In an example in FIG. 1, five stages 50A to 50E are defined. The number of the stages 50 may be smaller or larger than five.

The gap identification section 120 identifies a stage gap, which is a gap between two stages constituting a stage pair, for each pair of adjacent stages among the plurality of stages 50. The stage gap corresponds to, for example, a gap between a characteristic 60 of a lower stage and a characteristic 60 of a higher stage.

The reason/measure identification section 130 identifies a gap reason/shift measure from relationship information 160, for each stage pair. The gap reason/shift measure is at least one of a gap reason and a shift measure. The gap reason is a reason for existence of the identified stage gap and specifically, for example, at least one of an inhibitory/stimulatory factor, which will be described later, and a cognitive bias, which will be described later. The shift measure is a measure for causing a target person belonging to a lower stage in a stage pair to make a behavior change to shift to a higher stage in the stage pair. The relationship information 160 is information in which a relationship between a stage gap and a gap reason/shift measure is defined.

The processing execution section 140 executes result processing, which is processing related to the gap reason/shift measure identified for each stage pair.

According to the assistance system 100, the stage indicator 55 and the plurality of stages 50 following the stage indicator 55 are defined (generated) based on the behavior data 150 on the plurality of target persons as an origin, and at least one of a gap reason and a shift measure identified from a stage gap 60 between each pair of stages among the plurality of stages 50 thus defined is associated with the stage pair. In other words, a data set derived from the behavior data 150 obtained by measuring behaviors of the plurality of target persons and a data set derived from the relationship information 160 constructed by a different method from the method used to obtain the behavior data 150 are matched by the functions 110, 120, and 130 included in the assistance system 100, more specifically, the plurality of stages 50 created by analyzing the behavior data 150 and the gap reasons/shift measures obtained from the relationship information 160 are associated with each other. Accordingly, in each of various fields, even when no one has knowledge or know-how conversant in the field, it is possible to technically assist any target person who is not habituated to a behavior as a target in becoming habituated to the behavior as the target in the field.

One of specific examples of such technical assistance is as follows.

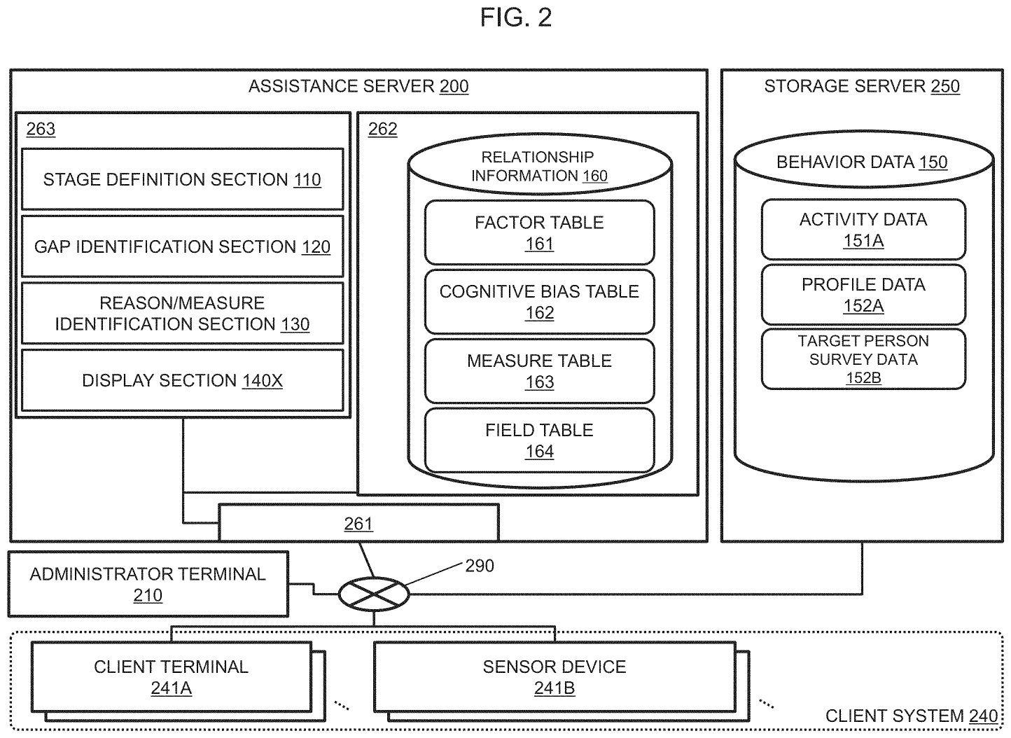

The behavior data 150 includes a plurality of types of quantitative data 151 and a plurality of types of qualitative data 152. The plurality of types of qualitative data 152 include, for example, target person survey data 152B (see FIG. 2), which is data indicating results of surveying the plurality of target persons (for example, respective records on a plurality of behaviors obtained by surveying the plurality of behaviors).

The stage definition section 110 classifies a plurality of quantitative data sets belonging to one or more types of the quantitative data 151 into a plurality of target person groups (clusters) 40, each of which is a group of target persons, (performs clustering) from a viewpoint determined based on at least one type of the qualitative data 152. In other words, as an example, the stage definition section 110 identifies a variable to be used in analysis from a qualitative viewpoint, and classifies target persons based on a result of analysis or predetermined quantitative data scores. The stage definition section 110 defines each of the plurality of stages 50 that follow the stage indicator 55 and into which the plurality of target person groups 40 are classified, based on a characteristic of each of the plurality of target person groups 40 and the above-mentioned survey answer data. According to the example in FIG. 1, the plurality of quantitative data sets are classified into five target person groups 40A to 40E, and the target person groups 40A to 40E are classified into the stages 50A to 50E, respectively.

As described above, since both a viewpoint of clustering and a viewpoint of the stage indicator 55 are determined based on at least one type of the qualitative data 152, it can be anticipated that the plurality of stages 50 that are defined based on a result of clustering and the stage indicator 55 are appropriate to a higher degree in a field to which the assistance system 100 is applied.

Note that the number of target person groups 40 classified into one stage 50 may be larger than one. In other words, for the plurality of stages 50 as a result of classification of the plurality of target person groups 40, the number of the stages 50 may be the same as, or larger than, the number of the target person groups 40.

The stage indicator 55 is defined by the stage definition section 110, based on one or more types of the qualitative data 152 and the target. Definition of the stage indicator 55 may be performed before clustering is started, or during clustering, or after clustering is finished (that is, after the plurality of target person groups 40 are determined). When the stage indicator 55 is defined before clustering is started, the clustering may be performed from a viewpoint following the stage indicator 55. In other words, the plurality of stages 50 may be defined along data that can be visualized as results of behaviors as an axis (the “axis” here is the “stage indicator 55”).

Characteristics of the target person groups 40 may be characteristics from one or more viewpoints such as generation, gender, job category, role, basic personality, and sense of values to a specific matter of target persons.

For each stage pair, the gap reason is at least one of an inhibitory/stimulatory factor and a cognitive bias. The inhibitory/stimulatory factor is at least one of an inhibitory factor and a stimulatory factor.

“Inhibitory factor” is a factor that inhibits a behavior change to shift from a lower stage to a higher stage. The inhibitory factor is, for example, a factor obtained based on an answer of a target person who belongs to a lower stage and does not shift to a higher stage (and/or a target person who has never shifted to the higher stage), among answers indicated by the above-mentioned answer data.

“Stimulatory factor” is a factor that achieves at least one of stimulating a behavior change to shift from a lower stage to a higher stage and preventing a behavior change to shift from (leave) a higher stage to a lower stage. The stimulatory factor is, for example, a factor obtained based on at least one of an answer of a target person who shifts from a lower stage to a higher stage and belongs to the higher stage (and/or a target person who has shifted to the higher stage before) and an answer of a target person who has shifted from (left) a higher stage to a lower stage before, among the answers indicated by the above-mentioned answer data.

The “cognitive bias” corresponds to a higher-order concept of the inhibitory/stimulatory factor (at least one of the inhibitory factor and the stimulatory factor) and is a bias defined based on expertise in behavioral science. The cognitive bias is a concept identical or similar to, or broader than, so-called “nudge”.

The relationship information 160 includes a factor table 161, which is an example of first information in which a relationship between a stage gap and an inhibitory/stimulatory factor is defined, a cognitive bias table 162, which is an example of second information in which a relationship between an inhibitory/stimulatory factor and a cognitive bias is defined, and a measure table 163, which is an example of third information in which a relationship between a cognitive bias and a shift measure is defined (each of the tables 161 to 163 will be described later).

The reason/measure identification section 130 performs (a) to (c) below, for each stage pair:

(a) Identifying an inhibitory/stimulatory factor 70 corresponding to a stage gap between the stage pair from the factor table 161;
(b) Identifying a cognitive bias 80 corresponding to the identified inhibitory/stimulatory factor 70 from the cognitive bias table 162; and
(c) Identifying a shift measure 90 corresponding to the identified cognitive bias 80 from the measure table 163.

According to the procedure of (a) to (c), which is an example, advantages as follows can be anticipated. Specifically, in the relationship information 160, an inhibitory/stimulatory factor and a shift measure are associated with each other via a cognitive bias. A cognitive bias is a systematic bias in human thinking or decision, including non-rationality of humans, based on expertise in behavioral science and, in other words, can also be said to be an actual nature of an inhibitory/stimulatory factor. If the actual nature of an inhibitory/stimulatory factor is known, it can be thought that an appropriate shift measure can be easily determined. Accordingly, for each stage pair, a shift measure can be anticipated that is appropriate for a stage gap between two stages constituting the stage pair.

Note that the reason/measure identification section 130 may perform at least one of the following processing, instead of the above-described (a) to (c).
For at least one stage pair, (b) and (c) may be transposed. For example, for at least one stage pair, the reason/measure identification section 130 identifies a cognitive bias for each of the stages by using a characteristic of each stage as a key and, based on the cognitive bias for each of the stages, identifies an inhibitory/stimulatory factor for the stage pair. In such a case, in the relationship information 160, the cognitive bias table 162, which is an example of the second information in which a relationship between an inhibitory/stimulatory factor and a cognitive bias is defined, may include information in which a relationship between an inhibitory/stimulatory factor and a combination of a cognitive bias for a lower stage and a cognitive bias for a higher stage is defined.
For at least one stage pair, the reason/measure identification section 130 may identify one of an inhibitory/stimulatory factor and a cognitive bias as a gap reason from a stage gap, and may identify a shift measure without identifying the other as a gap reason.
For at least one stage pair, the reason/measure identification section 130 may identify a shift measure from a stage gap without identifying any of an inhibitory/stimulatory factor and a cognitive bias as a gap reason. In such a case, the relationship information 160 may include information in which a relationship between a stage gap and a shift measure is defined, instead of, or in addition to, at least one of the tables 161 to 163.

Hereinafter, the present embodiment will be described in detail.

FIG. 2 shows an example of an entire configuration of a system according to the first embodiment.

A client system 240, a storage server 250, an administrator terminal 210, and an assistance server 200 are coupled to a communication network (for example, the Internet) 290. At least the assistance server 200 of the storage server 250, the administrator terminal 210, and the assistance server 200 is a constituent component of the assistance system 100.

The client system 240 includes one or more client apparatuses 241. For the one or more client apparatuses 241, a client terminal 241A such as a personal computer may be adopted, or various sensor devices 241B may be adopted. The client system 240 transmits at least one type of data obtained by measurement about a plurality of target persons to the storage server 250. The at least one type of data, or the data processed, is at least part of the behavior data 150.

The storage server 250 stores the behavior data 150. The behavior data 150 includes activity data 151A as an example of the quantitative data 151, and includes profile data 152A and target person survey data 152B as examples of the qualitative data 152. The activity data 151A is quantitative data obtained by measuring various activities of the plurality of target persons and may include, for example, human body measures, sensor data, mechanical data, PC operation logs, communicational information such as e-mails and SNS (Social Networking Service), image information (still images or moving images) shot by a camera such as a security camera, history information such as a purchase histories and work histories, management information created for work management, work-time schedule information, audio data, locating information (for example, information indicating positions located by using infrared, Wi-Fi®, UWB (Ultra Wide Band)®, or the like), and like data. The activity data 151A may include two or more types of the quantitative data 151. The profile data 152A includes, for each of the plurality of target persons, a data set indicating a profile (for example, age, gender, and the like) of the target person. The target person survey data 152B includes, for each of the plurality of target persons, a data set indicating a result of a survey (for example, a plurality of behaviors) (for example, respective track records of the plurality of behaviors), such as a data set indicating a result of an questionnaire (a plurality of questions) (respective answers to the plurality of questions) or a data set indicating a result of an interview (a plurality of questions) (respective answers to the plurality of questions).

The administrator terminal 210 may be an information processing terminal (for example, a personal computer) functioning as a client (for example, an input-output console) of the assistance server 200. The administrator terminal 210 is used by an administrator. “Administrator” may be an administrator of the assistance server 200, or a manager who manages the plurality of target persons. The administrator may be a target person.

The assistance server 200 includes an interface apparatus 261, a storage apparatus 262, and a processor 263 coupled to the interface apparatus 261 and the storage apparatus 262. The interface apparatus 261 is coupled to the communication network 290.

The storage apparatus 262 stores, for example, the relationship information 160 and one or more programs (not shown) for implementing the stage definition section 110, the gap identification section 120, the reason/measure identification section 130, and a display section 140X by being executed by the processor 263. The display section 140X is an example of the processing execution section 140. The relationship information 160 includes, for example, a field table 164 in addition to the factor table 161, the cognitive bias table 162, and the measure table 163 mentioned above.

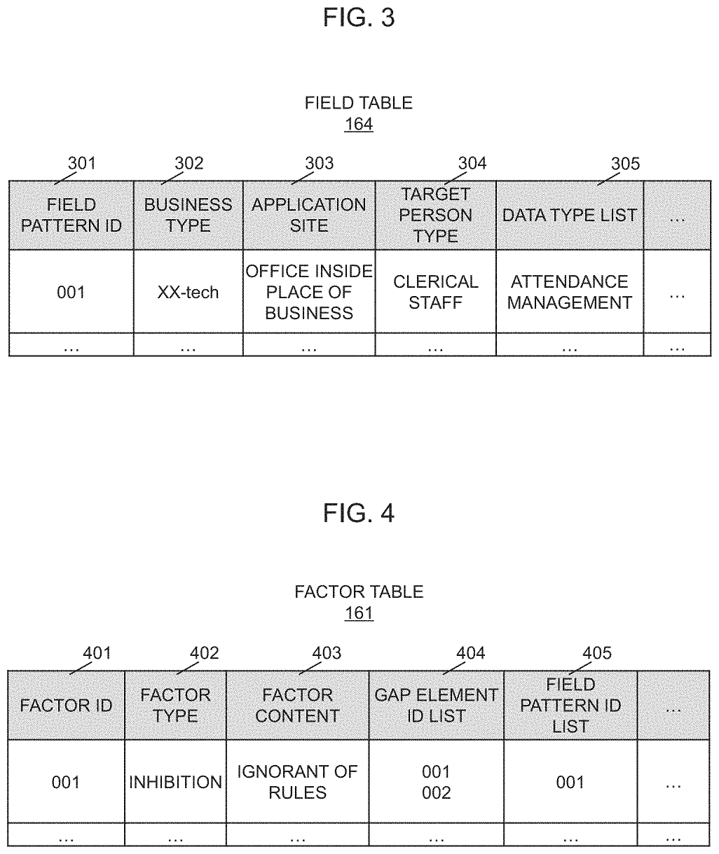

FIG. 3 shows an example of a structure of the field table 164.

The field table 164 retains information related to a field pattern. “Field” here corresponds to a collective term of various elements belonging to a “field” to which the assistance system 100 is applied. Examples of the elements belonging to a field include a business affair, an office, a public site, a living environment, and the like to which the assistance system 100 is applied. “Field pattern” is a pattern of such an element.

For example, the field table 164 includes an entry for each field pattern. For example, each entry retains information such as a field pattern ID 301, a business type 302, an application site 303, a target person type 304, and a data type list 305. Hereinafter, one field pattern will be taken as an example (“field pattern of interest” in the description regarding FIG. 3).

The field pattern ID 301 indicates an ID of a field pattern of interest. The information 302 to 305 is an example of information indicating constituent elements of the field pattern of interest. The business type 302 indicates a type of a business of application. The application site 303 indicates a site of application (an example of a site belonging to the business). The target person type 304 indicates a type of a target person of application (for example, a type derived from a profile, such as a job category). The data type list 305 indicates a list of types of data that can be used for objects of analysis in the behavior data 150.

FIG. 4 shows an example of a structure of the factor table 161.

For example, the factor table 161 includes an entry for each inhibitory/stimulatory factor. For example, each entry retains information such as a factor ID 401, a factor type 402, a factor content 403, a gap element ID list 404, and a field pattern ID list 405. Hereinafter, one inhibitory/stimulatory factor will be taken as an example (“inhibitory/stimulatory factor of interest” in the description regarding FIG. 4).

The factor ID 401 indicates an ID of an inhibitory/stimulatory factor of interest. The factor type 402 indicates which one of an inhibitory factor and a stimulatory factor the inhibitory/stimulatory factor of interest is. The factor content 403 indicates a content of the inhibitory/stimulatory factor of interest. The gap element ID list 404 indicates a list of IDs of gap elements applicable to the inhibitory/stimulatory factor of interest. The field pattern ID list 405 indicates a list of IDs of field patterns corresponding to the inhibitory/stimulatory factor of interest.

“Gap element” here is each of one or more elements included in a stage gap. For example, in each stage pair, each of two respective characteristics 60 (see FIG. 1) of two stages 50 included in the stage pair may have one or more characteristic elements. Each characteristic element may be a set of a characteristic item and a characteristic value of the item. The two characteristics 60 may have one or more common characteristic items. Accordingly, for each same characteristic item, it can be obtained, as a result of comparison between the two characteristics 60, whether or not the characteristic values are the same and, when the characteristic values are not the same, what difference lies. A stage gap may correspond to a set of characteristic elements having differences between the two characteristics 60, and a gap element may correspond to a characteristic element having a difference. A specific example is as follows. Specifically, characteristic elements (“characteristic item: characteristic value”) that are present in each stage can be “time: ‘unpunctual’/‘punctual’”, “meal: ‘eat’/‘not eat’”, “oversleeping: ‘wake late’/‘wake on time’”, and the like, and an inhibitory/stimulatory factor can be obtained by comparing the characteristic values of each characteristic item. Note that one or some characteristic values may be a qualitative value (for example, an answer to a question). For example, two or more identical or similar answers to a same question may be regarded as the same answers. An inhibitory/stimulatory factor in the present embodiment may be a patterned factor.

According to the factor table 161 as described above, an inhibitory/stimulatory factor corresponding to a field pattern and a set of gap elements can be obtained. For example, for the inhibitory/stimulatory factor of interest to be identified, at least all of conditions below need to be satisfied. Conditions for identifying an inhibitory/stimulatory factor may be arbitrarily designed.
At least one (for example, all or a majority) of one or more gap elements obtained by comparing characteristics 60 matches with all, or one or some, of gap element IDs included in the gap element ID list 404 corresponding to the inhibitory/stimulatory factor of interest.
An ID of a field pattern to which the assistance system 100 is applied matches with any one of field pattern IDs included in the field pattern ID list 405 corresponding to the inhibitory/stimulatory factor of interest.

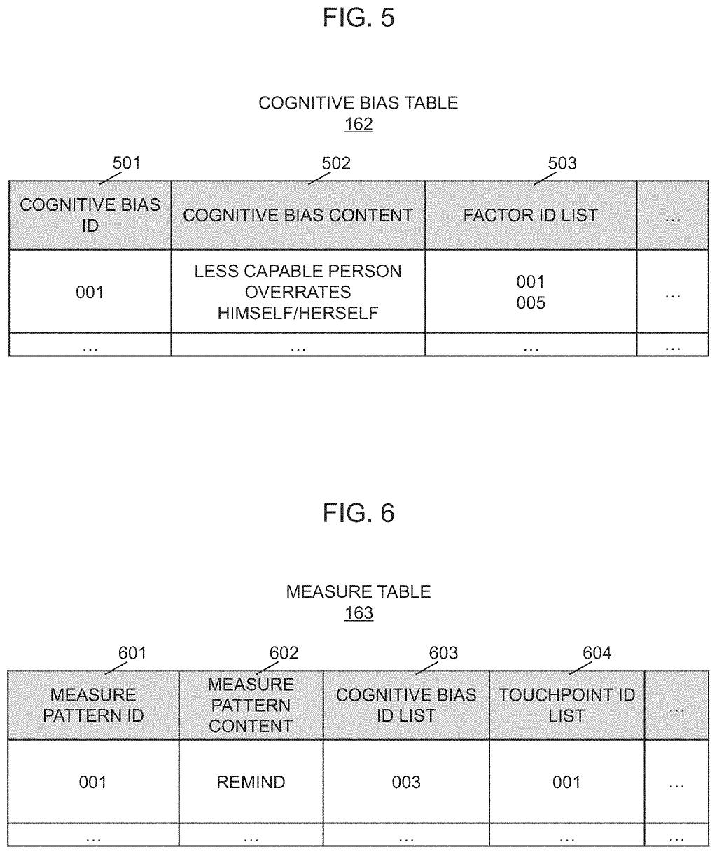

FIG. 5 shows an example of a structure of the cognitive bias table 162.

For example, the cognitive bias table 162 includes an entry for each cognitive bias. For example, each entry retains information such as a cognitive bias ID 501, a cognitive bias content 502, and a factor ID list 503. Hereinafter, one cognitive bias will be taken as an example (“cognitive bias of interest” in the description regarding FIG. 5).

The cognitive bias ID 501 indicates an ID of a cognitive bias of interest. The cognitive bias content 502 indicates a content of the cognitive bias of interest. The factor ID list 503 indicates a list of IDs of inhibitory/stimulatory factors belonging to the cognitive bias of interest.

FIG. 6 shows an example of a structure of the measure table 163.

For example, the measure table 163 includes an entry for each measure pattern. For example, each entry retains information such as a measure pattern ID 601, a measure pattern content 602, a cognitive bias ID list 603, and a touchpoint ID list 604. “Measure pattern” is a pattern of a shift measure. Hereinafter, one measure pattern will be taken as an example (“measure pattern of interest” in the description regarding FIG. 6).

The measure pattern ID 601 indicates an ID of a measure pattern of interest. The measure pattern content 602 indicates a content (for example, a label) of the measure pattern of interest. The cognitive bias ID list 603 indicates a list of IDs of cognitive biases for which it is preferable that at least one shift measure belonging to the measure pattern of interest be executed. The touchpoint ID list 604 indicates a list of IDs of touchpoints of shift measures belonging to the measure pattern of interest. In the present embodiment, “touchpoint” means a contact point between a shift measure and a target person.

Note that an entry corresponding to the measure pattern of interest may further retain information indicating one or more shift measures belonging to the measure pattern of interest. In such a case, the one or more shift measures belonging to the measure pattern of interest may be identified from the entry.

As described above, the tables shown in FIGS. 3 to 6 are examples of information included in the relationship information 160. With respect to the relationship information 160, the following may be adopted instead of, or in addition to, at least part of the foregoing.
An inhibitory/stimulatory factor (pattern) may be defined from a result of analysis of the target person survey data 152B.
A table for identifying one or more inhibitory/stimulatory factors by using an ID of a cognitive bias as a key may be prepared.
A plurality of inhibitory/stimulatory factors may be prepared beforehand for each field pattern, and the cognitive bias table 162 may be common regardless of field pattern.

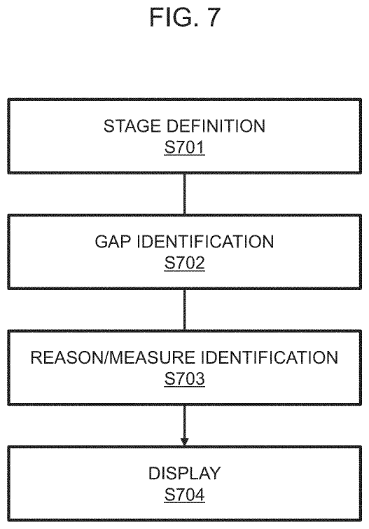

FIG. 7 shows an example of a flow of entire processing performed in the first embodiment. Note that in the description below, as an example of the plurality of stages 50, a plurality of stages of behavior change according to the Stages of Behavior Change Model will be adopted. Specifically, it is assumed that the five stages 50A to 50E illustrated in FIG. 1 are the behavior change stage 50A belonging to the pre-contemplation period, the behavior change stage 50B belonging to the contemplation period, the behavior change stage 50C belonging to the preparation period, the behavior change stage 50D belonging to the action period, and the behavior change stage 50E belonging to the maintenance period.

The stage definition section 110 performs stage definition (S701). The stage definition includes the above-described clustering, defining the stage indicator 55, and defining each of the stages 50A to 50E. With respect to S701, for example, the following may be adopted. In S701, a definition of the stage indicator 55 and a definition of each of the stages 50A to 50E are determined.
With respect to each stage 50 and each stage pair, a condition for identifying that the stage or the stage pair is applicable is predetermined for each qualitative data item (each data item in at least one type of the qualitative data 152) that can be adopted as a viewpoint of the stage indicator 55, in other words, for each stage indicator candidate (each candidate for the stage indicator 55).
A viewpoint (for example, a viewpoint derived from at least one type of the qualitative data 152) that can be adopted in clustering may be predetermined for each field pattern.
Clustering is performed, for example, for each viewpoint group (one or more viewpoints) corresponding to a specified field pattern. An arbitrary stage indicator candidate (a candidate for the axis) may be selected before clustering is started, and clustering may be performed based on the stage indicator candidate.
The stage definition section 110 performs condition determination as to whether or not a plurality of target person groups 40 obtained as a result of any clustering can be classified into the stages 50A to 50E such that a condition corresponding to an arbitrary stage indicator candidate is satisfied. When a result of the condition determination is true, the stage indicator candidate is defined (determined) as the stage indicator 55. When a result of the condition determination is false, the stage definition section 110 may select an arbitrary stage indicator from among stage indicator candidates yet to be selected, and condition determination as described above may be performed with respect to the stage indicator candidate. In other words, the stage definition section 110 may iterate selection of a stage indicator candidate and condition determination as described above until a result of the condition determination that is true is obtained.

The gap identification section 120 performs gap identification (S702). Specifically, for example, the gap identification section 120 identifies characteristics 60A to 60E corresponding to the stages 50A to 50E, respectively. For each stage 50, the characteristic 60 may be a characteristic of one or more target person groups 40 classified into the stage, and the characteristic may include a plurality of characteristic items and a plurality of characteristic values as described above. At least one characteristic item may be a question obtained from the answer data in the target person survey data 152B, and a characteristic value corresponding to the characteristic item may be an answer to the question.

The reason/measure identification section 130 performs reason/measure identification (S703). A specific example of the reason/measure identification is as described above.

The display section 140X performs display (S704). Specifically, as an example of the result processing, which is processing related to a gap reason/shift measure identified for each stage pair (for example, an inhibitory/stimulatory factor, a cognitive bias, and a measure pattern (and a shift measure belonging to the measure pattern) identified for each stage pair), the display section 140X performs processing of outputting information related to the identified gap reason and shift measure (which may include information related to the defined stages 50A to 50E). Although the output is a display (output of information for display) in the present embodiment, another type of output such as printing may be made instead of, or in addition to, to the display.

In the present embodiment, for example, the display section 140X displays at least one of a behavior design canvas sheet screen and a customer journey map screen, as a screen of the information related to the identified gap reason and shift measure.

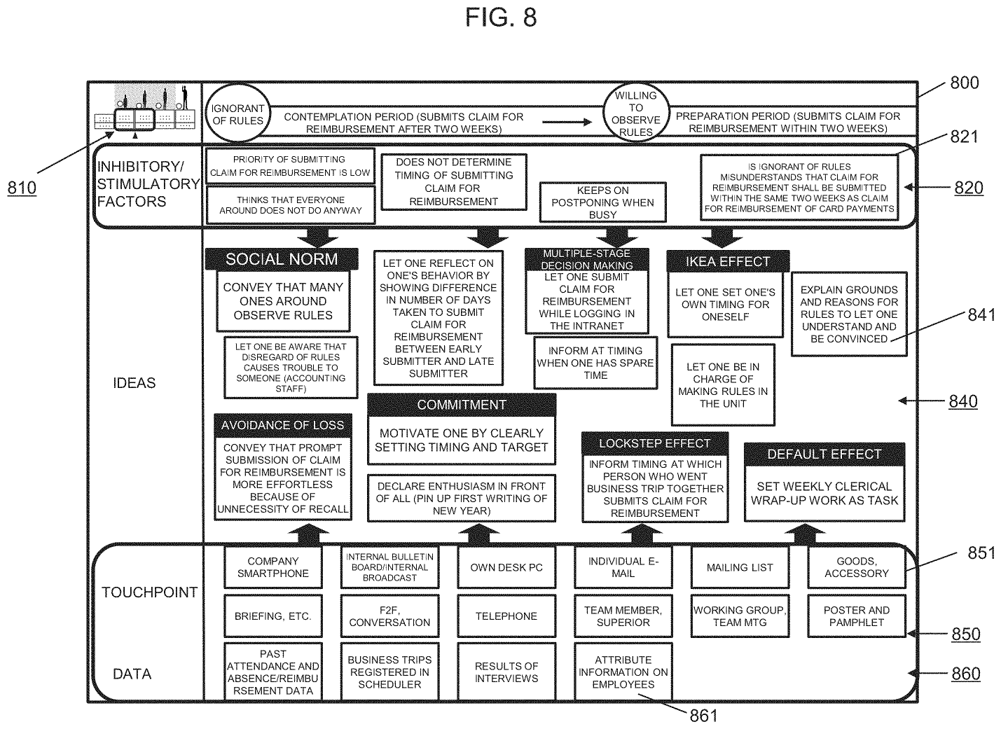

FIG. 8 shows an example of the behavior design canvas sheet screen.

A behavior design canvas sheet screen 800 is, for example, a GUI (Graphical User Interface). The screen 800 displays a behavior design canvas sheet. “Behavior design canvas sheet” is a sheet that shows, with respect to a stage pair specified by a user among the stages 50A to 50E, an identified gap reason, an identified shift measure, a touchpoint of the shift measure, and a label of data at least available with respect to the stage pair.

The behavior design canvas sheet screen 800 includes, for example, a stage area 810, a factor area 820, an idea area 840, a touchpoint area 850, and a data area 860. The rectangular areas 820 to 860 that are extensible in a horizontal direction (an example of a first direction) are arranged in a vertical direction (an example of a second direction orthogonal to the first direction).

The stages 50A to 50E are displayed in the stage area 810. A stage pair specified among the stages 50A to 50E (“stage pair of interest” in the description regarding FIG. 8) is highlighted. Thus, an administrator (an example of a user) can recognize information on which stage pair is displayed in the behavior design canvas sheet screen 800.

One or more factor objects 821 are displayed in the factor area 820. Each factor object 821 indicates a label (or a content) of an inhibitory/stimulatory factor identified with respect to the stage pair of interest.

One or more measure objects 841 are displayed in the idea area 840. Each measure object 841 indicates a shift measure belonging to any of one or more measure patterns identified with respect to the stage pair of interest, and a cognitive bias, if any, used in the shift measure.

One or more touchpoint objects 851 are displayed in the touchpoint area 850. Each touchpoint object 851 indicates a label (or a content) of a touchpoint.

The data area 860 shows one or more data objects 861. Each data object 861 indicates a label of a data type available or usable with respect to the stage pair of interest.

At least one of the objects 821, 841, 851, and 861 (and/or images of the plurality of stages displayed in the stage area 810) may be GUI parts. For example, when any one touchpoint object 851 is specified by the administrator, the display section 140X may highlights a measure object 841 of a shift measure applicable to a measure pattern to which a touchpoint corresponding to the object 851 belongs. Highlighting may be relative highlighting, for example, by changing a form of displaying the measure object 841, or making non-applicable measure objects 841 of the shift measure undisplayed.

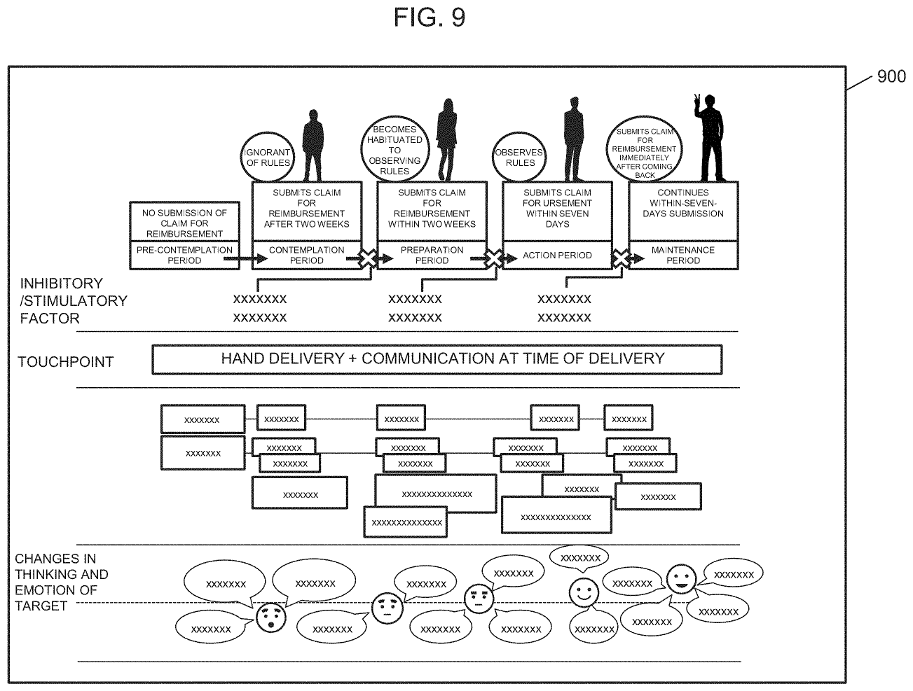

FIG. 9 shows an example of the customer journey map screen.

A customer journey map screen 900 is, for example, a GUI (Graphical User Interface). The screen 900 displays a customer journey map. “Customer journey map” is a sheet that shows a panoramic view of a plurality of stages, a gap reason identified with respect to each stage pair, and a shift measure identified with respect to each stage pair.

In any of the screens 800 and 900, at least part of the displayed information may be information identified from at least part of the behavior data 150 and the relationship information 160. Accordingly, of the information displayed in the screen 800 or 900, an information item that is not mentioned in the description of the behavior data 150 and the relationship information 160 may actually be included in any one of the behavior data 150 and the relationship information 160, or may be included in other information than the behavior data 150 and the relationship information 160.

The foregoing is the description of the first embodiment.

Note that after S701 to S703 are performed (for example, after S704 is performed thereafter), measurement (verification) of an effect of S701 to S703, that is, effect measurement such as comparison between the behavior data 150 before and after the displayed shift measure is conducted may be performed. Based on a result of the effect measurement, information update may be performed on information (typically, the relationship information 160) to be referred to in the processing in S701 to S703 by, for example, the stage definition section 110. The series of processing that is S701 to S703→effect measurement→information update as described above is iterated, whereby optimization of the relationship information 160 can be anticipated, and resultantly, optimization of matching between the behavior data 150 and the relationship information 160 (optimization of matching between the definitions of the plurality of stages 50 and the plurality of gap reasons/shift measures) can be anticipated.

### Second Embodiment

A second embodiment will be described. In the description below, differences from the first embodiment will mainly be described, and a description of common points with the first embodiment will be omitted or simplified.

In the second embodiment, selection and execution of a shift measure are performed instead of (or in addition to) display of the information related to a gap reason/shift measure (for example, a behavior design canvas sheet and a customer journey map).

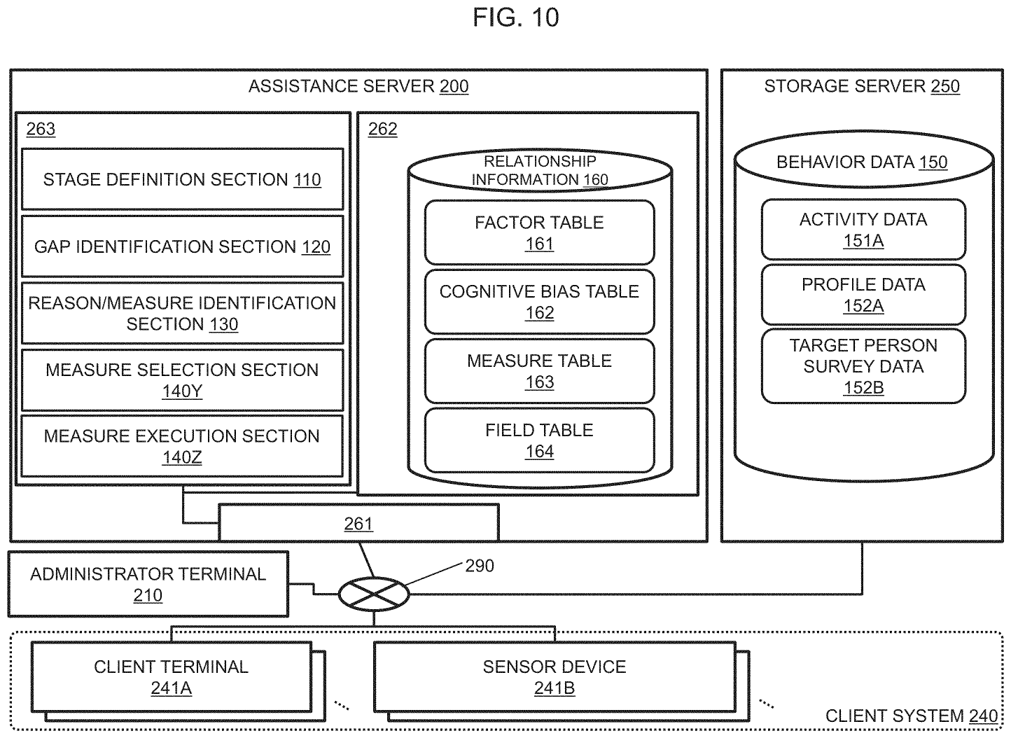

FIG. 10 shows an example of an entire configuration of a system according to the second embodiment.

In the assistance server 200, a measure selection section 140Y and a measure execution section 140Z are implemented instead of (or in addition to) the display section 140X. The measure selection section 140Y and the measure execution section 140Z are examples of the processing execution section 140. The measure selection section 140Y selects a shift measure to be executed from among shift measures identified by the reason/measure identification section 130. The measure execution section 140Z executes the shift measure selected by the measure selection section 140Y.

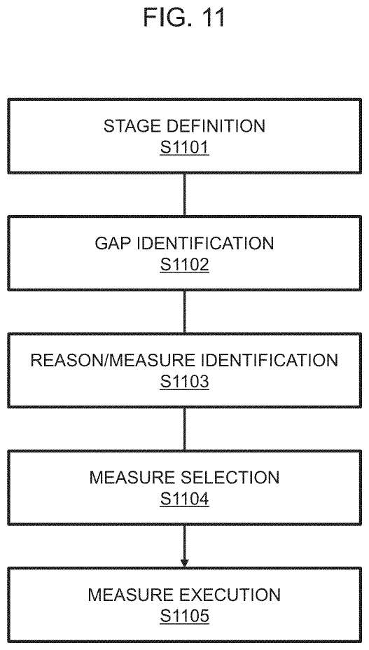

FIG. 11 shows an example of a flow of entire processing performed in the second embodiment.

Processing similar to S701 to S703 is performed (S1101 to S1103).

Thereafter, the measure selection section 140Y performs measure selection (S1104). Specifically, for example, with respect to each of one or more target persons among a plurality of target persons, the measure selection section 140Y selects a shift measure applicable to the target person from among shift measures identified by the reason/measure identification section 130 with respect to a stage pair including a stage to which the target person belongs and a next higher stage. Each of the “one or more target persons” may be a target person who is arbitrarily selected, or may be a target person on which profile-related information or the like satisfies a predetermined condition. The selected shift measure may be any one of the following.
In iteration of a series of processing that is S1101 to S1103→effect measurement→information update, the stage definition section 110 assigns, for each shift measure (or measure pattern) executed, a score according to a degree of an effect measured about the shift measure. The measure selection section 140Y selects a shift measure assigned a relatively high score (for example, a highest score) among the shift measures identified by the reason/measure identification section 130.
For each of the one or more target persons, the measure selection section 140Y selects a shift measure that is suitable to the profile-related information or the like on the target person, among the shift measures identified by the reason/measure identification section 130. In such a case, for example, in the measure table 163, information related to a target person (for example, profile-related information on a target person) may be associated with each shift measure.

The measure execution section 140Z performs measure execution (S1105). Specifically, the measure execution section 140Z executes the shift measure selected in S1104.

An example of the shift measure execution is output (for example, printing) of an individual report to a target person.

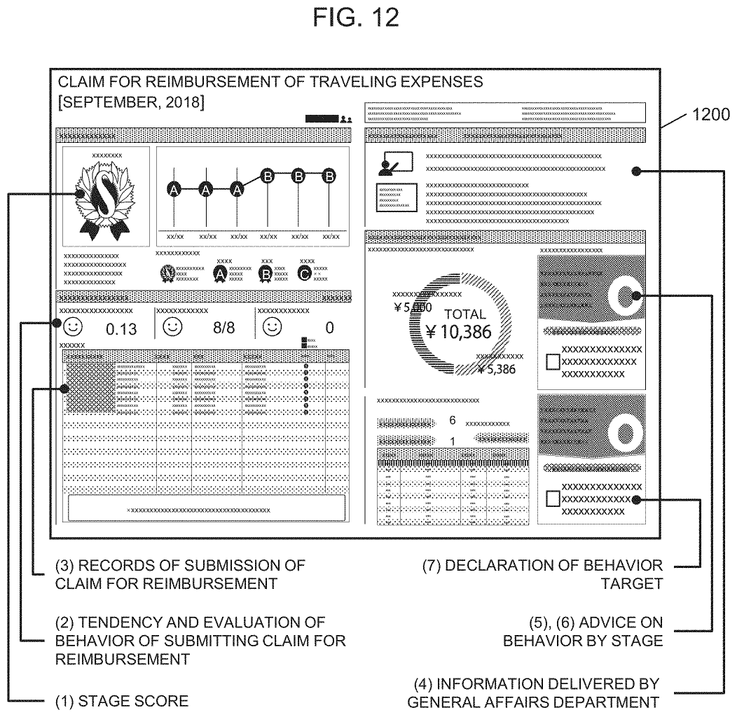

FIG. 12 shows an example of the individual report.

An individual report 1200 is a report made for a target person individually and is, for example, a report that shows information according to a gap reason and a shift measure applicable to the target person, among gap reasons and shift measures identified with respect to each stage pair.

According to the example in FIG. 12, the individual report 1200 is a “claim for reimbursement of traveling expenses” report on an individual employee, regarding accelerated submission by employees of a claim for reimbursement of traveling expenses.

Although several embodiments have been described hereinabove, the embodiments are given for illustrative purposes to describe the present invention, and are not intended to limit the scope of the invention to the embodiments. The present invention can also be implemented in other various forms.

What is claimed is:

1. A system assisting a target person in behavior change and habituation, comprising:
a stage definition section that analyzes behavior data including various data obtained by measuring behaviors of a plurality of target persons, and based on a result of analysis of the behavior data, defines a stage indicator, which is an indicator as a criterion of a plurality of stages via which a behavior as a target of habituation is aimed at step by step, and each of the plurality of stages following the stage indicator;
a gap identification section that identifies a stage gap, which is a gap between two stages constituting a stage pair, for each pair of adjacent stages among the plurality of stages;
a reason/measure identification section that, for each of the stage pairs, identifies a gap reason/shift measure that is at least one of a gap reason, which is a reason for existence of the identified stage gap, and a shift measure, which is a measure for causing a target person belonging to a lower stage to make a behavior change to shift to a higher stage, from relationship information in which a relationship between the stage gap and the gap reason/shift measure is defined; and
a processing execution section that executes result processing, which is processing related to the gap reason/shift measure identified for each of the stage pairs.

2. The system according to claim 1, wherein the various data includes a plurality of types of quantitative data and a plurality of types of qualitative data,
the plurality of types of qualitative data includes target person survey data, which is data indicating a result of a survey on the plurality of target persons,
the stage definition section classifies a plurality of quantitative data sets belonging to one or more types of the quantitative data into a plurality of target person groups, each of which is a group of target persons, from a viewpoint determined based on at least one type of the qualitative data, and defines each of the plurality of stages that follow the stage indicator and into which the plurality of target person groups are classified, based on a characteristic of each of the plurality of target person groups and the target person survey data, and
the stage indicator is defined based on one or more types of the qualitative data and the target.

3. The system according to claim 2, wherein with respect to each of the stage pairs, the gap reason is at least one of following matters:
an inhibitory factor, which is a factor inhibiting the behavior change to shift from a lower stage to a higher stage;
a stimulatory factor, which is a factor of at least one of stimulating the behavior change to shift from a lower stage to a higher stage and preventing a behavior change to shift(leave) from a higher stage to a lower stage; and
a cognitive bias, which corresponds to a higher-order concept of the inhibitory/stimulatory factor that is at least one of the inhibitory factor and the stimulatory factor, and which is defined based on expertise in behavioral science,
the relationship information includes first information in which a relationship between the stage gap and the inhibitory/stimulatory factor is defined, second information in which a relationship between the inhibitory/stimulatory factor and the cognitive bias is defined, and third information in which a relationship between the cognitive bias and the shift measure is defined, and
the reason/measure identification section identifies, for each of the stage pairs, the inhibitory/stimulatory factor corresponding to the stage gap between the stage pair from the first information, identifies the cognitive bias corresponding to the identified inhibitory/stimulatory factor from the second information, and identifies the shift measure corresponding to the identified cognitive bias from the third information.

4. The system according to claim 3, wherein the result processing is processing of outputting at least one of following matters:
a behavior design canvas sheet that shows, with respect to a stage pair specified by a user among the plurality of stages, the identified gap reason, the identified shift measure, a touchpoint of the shift measure, and a label of data at least available with respect to the stage pair; and
a customer journey map that shows a panoramic view of the plurality of stages, the gap reason identified for each of the stage pairs, and the shift measure identified for each of the stage pairs.

5. The system according to claim 3, wherein the result processing is processing of, with respect to each of one or more target persons among the plurality of target persons, selecting and executing a shift measure applicable to the target person, among shift measures identified with respect to a stage pair including a stage to which the target person belongs and a next higher stage.

6. A method for assisting a target person in behavior change and habituation, comprising:
by a computer, analyzing behavior data including various data obtained by measuring behaviors of a plurality of target persons, and based on a result of analysis of the behavior data, defining a stage indicator, which is an indicator as a criterion of a plurality of stages via which a behavior as a target of habituation is aimed at step by step, and each of the plurality of stages following the stage indicator;
by a computer, identifying a stage gap, which is a gap between two stages constituting a stage pair, for each pair of adjacent stages among the plurality of stages;
by a computer, for each of the stage pairs, identifying a gap reason/shift measure that is at least one of a gap reason, which is a reason for existence of the identified stage gap, and a shift measure, which is a measure for causing a target person belonging to a lower stage to make a behavior change to shift to a higher stage, from relationship information in which a relationship between the stage gap and the gap reason/shift measure is defined; and
executing result processing, which is processing related to the gap reason/shift measure identified for each of the stage pairs.
